(19) United States
(12) Patent Application Publication
(10) Pub. No.: US 2006/0127510 A1
(43) Pub. Date: Jun. 15, 2006

(54) Harvesting and pelletizing yew biomass for extraction of taxanes and other natural products

(75) Inventors: Rex T. Gallagher, Beverly, MA (US); Christopher D. Howe, Beverly, MA (US); Edward M. Desimone III, Indianapolis, IN (US); Brian A. Bucher, Somerville, MA (US); Barry J. Hand, Acton, MA (US); Steven L. Perich, Boulder, CO (US); Michael Aeschliman, Archbold, OH (US); Ivan Sparks Wyse, Archbold, OH (US)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Natural Pharmaceuticals, Inc.

(21) Appl. No.: 11/173,477

(22) Filed: Jun. 30, 2005

Related U.S. Application Data
(63) Continuation-in-part of application No. PCT/US03/41824, filed on Dec. 30, 2003.
(63) Continuation-in-part of application No. PCT/US03/41774, filed on Dec. 30, 2003.

(51) Int. Cl. A61K 36/19; C07D 305/14

(52) U.S. Cl. 424/769; 264/109; 549/510

(57) ABSTRACT

A process for harvesting yew biomass and a process of making Yew pellets from Yew tree material is disclosed herein. The process for harvesting comprising the steps of (i) harvesting the tops or roots of the Yew tree; (ii) washing the harvested material; (iii) drying the harvested material; and (iv) grinding the harvested material into a fine powder. The process for making Yew pellets comprising the steps of (i) adding from about 1% to about 20% w/w water to the Yew tree material; and (ii) compressing the Yew tree material in a die to form a pellet whereby the resulting pellet has a final moisture content of less than 10% by weight. The yew pellets produced herein are suitable for extracting taxane molecules from the pellets.

### CROSS-REFERENCE TO RELATED APPLICATIONS

This application is a continuation-in-part of PCT/US03/41824, filed Dec. 31, 2003, and PCT/US03/4177, filed Dec. 30, 2003. These prior applications are hereby incorporated herein by reference in their entirety.

This patent application also claims the benefit of U.S. Provisional Appl. No. 60/437,237, filed Dec. 31, 2002, and U.S. Provisional Appl. No. 60/437434, filed Dec. 31, 2002. Both of these prior applications are incorporated herein by reference in their entirety.

### BACKGROUND OF INVENTION

The present invention pertains to harvesting, washing, chopping, cutting drying, grinding and pelletizing biomass tree material for extraction of taxanes and other natural products.

Yew trees contain a family of natural diterpenoid compounds called taxanes. One of these taxanes in particular, taxol, has been developed as a major anticancer drug which successfully treats a number of human cancers. The name taxol was given to the compound by the original discoverers of this material, Wani & Wall. Subsequently the name was registered as a trademark, and the compound is now referred to in the scientific literature as ‘Paclitaxel.’ However, a large body of scientific and industry publications still refer to the natural compound as taxol.

Large-scale extraction of plant biomass has been practiced, and a number of patents have been granted for various extraction and purification processes. These patents generally fall into two categories: (1) animal feed; and (2) fuel. For example, U.S. Pat. No. 6,375,447 is directed to a feed pellet mill that has one inlet for the feed, one outlet for the pellet, and one outlet for water expressed from the forming pellet. This allows for excess water to beremoved from the biomass as the pellet is being formed. U.S. Pat. No. 4,613,339 is directed to a process that pelletizes spent sorghum residue, “bagasse,” by storing and then pelletizing the residue. U.S. Pat. Nos. 5,682,683 and 5,728,447 describes process for forming pellets of grains and cereals with a waved-brick structure, providing the pellet with better handling properties.

Paclitaxel was initially discovered in the bark of the Pacific Yew tree. A number of patents described processes for removing the bark from the trunk-wood of the tree. For example, U.S. Pat. No. 3,826,433 describes a process for separating the bark from the shipped wood through steam, mechanical compression, milling, and screening. U.S. Patent Application US2002/0114853 describes the use of pelletization in the extraction of a variety of compounds (including taxanes) using selective extraction of components using acidic and basic solvents from Taxus yunnaneusis bark, or Yew tree needles. Other patents that are relevant to extractions from biomass may use powdered, pulverized, or shredded material (See U.S. Pat. Nos. 6,1751,035 B1, 6,264,998 B1, 6,392,070 B1).

Yew is a name ascribed to a number of trees which are Taxus species; Taxus being the main genus in the family Taxaceae. Originally isolated from the bark of the Pacific Yew (Taxus brevifolia) collected from Washington State, beginning in 1962, taxol was subsequently reported as occurring in two other Taxus species, including Taxus baccata (European Yew) and Taxzts cuspidata (Japanese Yew), in 1971, Following intensive investigations, taxol was further reported to occur in a number of other Taxus species and cultivars. These include, but are not limited to: Taxus globosa (Mexican Yew), Taxus floridana (Florida Yew), Taxus canadensis (Canadian Yew), Taxus wallichiana (Himalayan Yew), Taxus yunnanensis, Taxus chinensis, and also a number of ornamental hybrids, such as Taxus media cultivars, e.g.: T.nzedia ‘Densiformis’, T.media ‘Hicksii’, T.media ‘Brownii’, T.media ‘Dark Green Spreader’, T.niedia ‘Runyan’, T.rnedia ‘Wardii’, T.media ‘Tautonii’,T cuspidata ‘Capitata’, etc.

In the present invention, Yew biomass material may be derived from any Taxus species, including but not limited to the species and cultivars described above. Other Taxus species for use in the present invention are identified in: Chadwick, L. C. and Keen, R. A. May 1986, “A study of the Genus Taxus”, Res. Bull. 1086, Ohio Agricultural Research and Development Center; Appendino, G. 1995, “The Phytochemistry of the Yew Tree”:Phytochemistry, Natural Products Reports 12(4): 349-360; Convention On International Trade in Endangered Species of Wild Fauna and Flora: Eleventh meeting of the Plants Committee, LangKawi (Malaysia), 3-7 Sep. 2001, Document PC 11 DOC. 22-p.1, United States of America; and Greer, C. E., Schutzki, R. E., Fernandez, A. and Hancock, J. F. October/December 1993, “Electrophoretic Characterization of Taxt1S Cultivars”: HortTechnology, 3(4): 430-433, Each of these references are incorporated herein by reference in their entirety.

Therefore, in one alternative embodiment, the starting material for this invention may be derived from a plant material selected from the group of plants commonly referred to as Yew trees. Suitable plants of this group are species of Taxus. Among Taxus species, Taxus x media cultivars are preferred. For example, preferred cultivars include, but are not limited to, T.media ‘Hicksii’ or T.media ‘Dark Green Spreader’. While it is convenient to use certain parts of the Yew tree in this invention, the Yew biomass for pelletization can be derived from the whole plant or from separated parts such as wood, stems, roots, leaves (needles), seeds, or any combination thereof. The material to be pelletized are derived from the bark or the needles are used. In one preferred embodiment, the material to be pelletized is dry ground yew tree root biomass (powder).

The large-scale processing of Yew tree biomass typically involves some kind of sizereduction processing. This is done to produce a more-or-less homogeneous ground powder which can be extracted with organic solvents in suitable, large-volume containers. Problems arise with handling the ground biomass material, particularly with transportation, safety, and ease and efficiency of solvent extractions. Solvent extraction problems arise from poor solvent contact due to channeling, ‘clumping’, or poor solvent circulation.

In the present invention, pellets can be readily made from ground biomass materials using commercially available pelletizing mills. Suitable large-scale pelletizing machinery is available from various sources, which include California Pellet Mill Co, Crawfordsville, Indiana; Sprout-Bauer, Muney, Pa.; Bliss Industries, Ponca City, Okla. Typically, very large and heavy duty machinery is used for the process of pelletizing biomass materials such as hay, alfalfa, etc. The machinery used usually involves engines that can range in capacity up to 10 tons/hour throughput which utilize massive engines of several hundred HP. The associated drying and mass transporting systems involve large facilities and equipment, often including large storage sheds, silos, semitrailer and rail wagon transportation, etc.

The advantages of using pelletized Yew biomass materials over non-pelletized materials include but are not limited to the following: (i) ease of bulk handling; (ii) shipping/transportation/storage; (iii) freedom from dust (cleaner, safety factor, environmental factor, reduced product loss); (iv) greater density of solids leads to less volume required in shipping and storage; (v) greater density of solids requires smaller processing vessel volumes; (vi) non-packing nature of pellets resists volume-unloading issues after transport (flour can pack into a cement); (vii) larger size of pellets allows for greater range of equipment use in processing (larger filters); (viii) uniform size-diameter distribution vs. ground biomass; (ix) extraction of biomass lends itself to faster/easier operation due to non-packing nature of the pellets (higher throughput of solvent through bed of pellets); (x) use of pellets decreases channeling and loss of contact between pellets and extraction solvent; (xi) use of pellets decreases pockets of biomass with low solvent-turnover rates; (xii) stability of product, including reduced oxidation due to smaller surface area exposed to the atmosphere; reduced microbial fermentation problems (toxins, heat); and (xiii) use of continuous extraction equipment including automated counter-current extraction equipment (e.g. Crown Extractors).

In one alternative embodiment of the present invention, it was discovered that pelletizing ground powdered Yew biomass offers substantial advantages in the handling, processing and, most importantly, extraction of taxanes and other material products from the biomass. In particular, efficiency and ease of solvent extraction is a major advantage in processing the pelletized Yew biomass. Thus, the present invention provides a process for manufacturing pelletized Yew material without significant reduction of the available taxane content in the biomass. Also, such process does not adversely impact the extraction process, such that additional purification and/or extraction steps are required. Further, in another alternative embodiment, the present invention provides stable pellets from ground, dried Yew roots, at both the laboratory scale and at the industrial scale. No significant loss/degradation of extractable paclitaxel resulting from the present pelletization process was observed.

### SUMMARY OF THE INVENTION

It is an object of the present invention to provide a process for harvesting and processing Yew biomass for extracting taxanes and other natural products. Another object of the present invention is to provide an efficient and economical process for harvesting and processing yew biomass, including producing ground material suitable for pelletization.

Another object of the present invention is to provide a process for making pellets from ground Yew tree material (including ground material) produced by the process described herein. It is an object of the present invention to provide a process for producing pelletized Yew biomass for extracting taxanes and other natural products.

Another object of the present invention is to provide an efficient and economical process for producing Yew pellets from Yew tree material, including ground material (powder).

Another object of the present invention is to provide a process for making pellets from Yew tree material (including powdered material) which are easily handled, stored, and used.

A further object of the present invention is to provide a process for making Yew pellets from Yew tree material (including powdered material) wherein the resulting pellets have appropriate amounts of water for the extraction process.

A further object of the present invention is to provide a process for making Yew pellets with appropriate amounts of water to minimize undesirable microbal growth of the pelletized material.

In one alternative embodiment, the foregoing objects and advantages of the invention are achieved in accordance with the present invention in which yew biomass material is produced from, for example, Yew tree material, or a mixture of Yew tree materials, by harvesting, washing, grinding, drying and pelletizing the biomass material. While the invention may be utilized in the processing of various raw materials, it has been found especially useful in converting harvested Yew into finely ground material suitable for pelletizing.

In one alternative embodiment, the present invention is directed to making Yew pellets from Yew tree material. The process comprising the steps of (i) adding from about 1% to about 20% w/w water to ground Yew tree material; and (ii) compressing the Yew tree material in a die to form a pellet. In one embodiment, the resulting pellet has a final moisture content of less than 10% by weight. The Yew pellets produced herein are suitable for extracting taxane molecules from the pellets.

In one alternative embodiment, the foregoing objects and advantages of the invention are achieved in accordance with the present invention in which a pelletized product is produced from, for example, a finely ground pelletizabie Yew tree material, or a mixture of finely ground Yew tree materials and water, by pressing the material through appropriate die openings in a commercial or laboratory scale pelletizer such as, for example, a California pellet mill, or a Bliss pellet mill. While the invention may be utilized in the processing of various raw materials, it has been found especially useful in producing a pelletized Yew from a finely ground material.

These and other objects and advantages of the present invention will become more readily apparent from the following detailed description.

### BRIEF DESCRIPTION OF THE FIGURES

FIG. 1 is a non-limiting, schematic representation of one alternative embodiment of the present invention.

FIG. 2 is a non-limiting, schematic representation of one alternative embodiment of the present invention.

### DETAILED DESCRIPTION OF THE INVENTION

1. Harvesting Yew Tree Roots

Biomass harvesting of Yew trees can be carried out on two main plant biomass components:

1) the below-ground part of the trees, i.e. the roots of the trees, including one or more of the following plant parts: roots, root bark, stump and/or

2) the above-ground part of the trees, including one or more of the following plant parts: needles, stems, branches, bark, trunk.

The method of harvesting will differ in a number of aspects, depending on which of these two components is harvested. In the case of below-ground parts, a key requirement for processing the removal of the biomass from the ground is removal of excess soil. Soil typically adheres to the harvested biomass in significant amounts. Typically, Yew trees are often grown in sandy soils, due to the good drainage properties of such soils. The presence of significant quantities of soil/sand adhering to roots and below-ground components however provide a challenge for subsequent machine processing. The high silica content of sand causes it to be very abrasive on cutting and grinding equipment. This, in turn, causes excessive wear and loss of performance, downtime, and failure of chopping, cutting, grinding, and pelletizing machinery.

Tops of Yew plants, including needles, can be harvested with various types of equipment. In one alternative embodiment, a harvesting procedure is as follows: a tractor fitted with a suitable agricultural chopper, such as a Kemp Head corn chopper, chops the tree parts to approx. I″ pieces, which are then blown into a mobile hopper that follows the tractor. The loaded hopper may be loaded into a large trailer which then transports the chopped biomass directly to the drying or pelletizing facility. It is desirable that the chopped tops are not subject to high temperatures while being transported, and awaiting drying or pelletizing, as this can cause significant loss of valuable taxanes in the biomass material. In a preferred transportation mode, the chopped biomass can be transported in chilled transportation trailers.

To begin the process of obtaining raw material, the Yew plants must be harvested from the field on which they are grown. The biomass is processed in two main streams: tops (the part of the plant that exists aboveground) and roots (everything below the leafing portion of the plant). The roots contain a higher grade of raw material, where the taxane content is slightly greater than in the tops, which contain a greater amount of waxes and other hydrophobic compounds that add to the difficulty of recovering raw material.

In one alternative embodiment of the present invention, the process of harvesting the tops begins with cutting the tops off of the plant. The preferred equipment is a Kemper head chopper (used for corn harvesting). The tops of the plants are processed immediately through a grinder, and then the ground plant is blown into a hopper that is towed behind the harvesting equipment. This process removes the tops from the plants for later processing, leaving the roots in the ground. Other, more preferred equipment may include a biomass harvester as used in the fuel industry.

In another alternative embodiment, a biomass harvester designed for the reduction of shrubbery to mulch, and developed by Dr. Peter Felker at Texas A&M (Kingsville, Tex.), can be used in the present invention. This is a flail harvester adapted for cutting trunks of up to 10 cm in diameter. The harvesting rate was measured using mesquite shrubbery at over 7000 kg /h of fresh-weight biomass, or 1.0 hectare/hour (2.3 acres/h). This equipment is available from the Brown Bear corporation (P.O. Box 29, 602 Ave. of Industry, Corning, Iowa USA 50841) with a harvesting rate of 1.2 hectare/h (3 acres/h), and a trunk diameter capacity of up to 25 cm. This equipment has the potential to contribute largely to the economics of the biomass harvesting process.

The remaining roots of the taxes plants are plowed from the ground, using a modified undercutter, originally designed as a carrot or potato digger by Vogel Engineering (W227 N913 Westmound Dr. Waukesha, Wis. 53186). The undercutter is hauled across the fields using a four-wheel drive tractor. The roots are then loaded onto hoppers using Kabota minicranes. The hoppers are placed on trailers that are pulled by tractors to the washing area. If the harvesting area is far from the washing area, larger trailers (walking floor or dump) may be used.

An extensive range of equipment is available to carry out the harvesting and subsequent processing of Yew biomass. A list of suitable equipment is set forth in Table 1 below:

TABLE 1
HARVESTING

| ACTIVITY | EQUIPMENT |
|---|---|
| Harvesting Tops from Fields: A tractor | Tractor with Kemper (or |
| equipped with a Kemper Head (corn | similar head)′ |
| chopper) is used to harvest the tops | Tractor with hopper |
| of the trees. The trees are initially | Walking floor or dump |
| chopped by the Kemper head and then | trailer with truck |
| ground to approximately 1″ pieces. | |
| The tops are then blown into a hopper | |
| that trails the tractor. Once the | |
| hopper is full it is loaded into a | |
| large trailer (walking floor or dump). | |
| The tops are sent directly to the | |
| dryer/pelletizer. Pre-grinding is not | |
| necessary prior to drying. | |
| Removal of Tops: Tops are removed prior | Brush Hog |
| to harvesting roots. This is accomplished | |
| by using the tops harvesting method | |
| or by mowing the tops to ground level by | |
| using a brush hog. | |
| Harvesting Roots from Fields (Primary | 1 Large Undercutter |
| Harvest Method): A specially designed | 1 Hopper and Tractor |
| large undercutter is used to harvest the | |
| roots. The large undercutter was designed | |
| by Vogel Engineering and appears to be a | |
| modified potato or carrot digger. The | |
| roots are loaded into a hopper that is | |
| pulled by a tractor. This is the preferred | |
| method for harvesting, but the Lundale | |
| Undercutters may be used for smaller jobs. | |
| Harvesting Roots from Fields (Alternate | 3-4 Lundale Undercutters |
| harvest method): 3 to 4 Lundale | 3-4 4 Wheel Drive tractors |
| Undercutters are used to pull roots from | 1 Trailer and tractor |
| the ground (Undercutters are pulled by | 2 Kabota mini-cranes |
| 4 wheel drive tractors). The roots are | |
| then loaded onto a trailer, which is | |
| pulled by a tractor, with 2 Kabota | |
| mini-cranes. | |
| Transport of Roots to Washing Station | Trailer and tractor |
| The harvested roots are transported to | Walking floor or dump |
| the washing station on the trailers. | trailers pulled by truck. |
| Large trailers (walking floor or dump) | |
| are used in situations where the | |
| harvesting location is several miles | |
| away from the wash station. | |

2. Washing the Harvested Material

To overcome the problem of abrasive wear on processing equipment, it is necessary to incorporate a washing step immediately post-harvest in order to remove as much soil/sand as possible from the biomass. A requirement of the washing process is that it must not however severely damage roots, or cause loss of root bark, as this can result in loss of valuable natural components, which are sometimes at higher content in the root bark than the rest of the root biomass.

In particular, loss of root bark in an aggressive washing process can result in significant losses of paclitaxel and other desired taxanes. Further, mechanical damage to harvested roots in an aggressive washing protocol can activate enzyme systems which can cause loss of paclitaxel and taxanes. Also, excess heating and caustic wash protocols must be avoided as these can result in loss of paclitaxel and desired taxanes.

In one effective method, harvested roots are transported quickly to a washing station. A large crane with ‘lifting tongs’ (or equivalent grappling device) is used to dunk the roots in a series of two steel tanks filled with cold water. The tanks typically measure 10 ft×30 ft×6 ft high. The roots are lifted from the second tank after the final dunking by the crane, and sprayed with a cold water stream from a pump, typically of 40 HP, to thoroughly wash out clinging soil/sand from the clumps of root biomass. The washed roots are then staffed, and after drying (½0 to 1 day, ambient atmosphere), the dried material is loaded on a suitable transporter and transported to a chopping/drying/grinding/pelletizing facility station.

In another alternative embodiment, using a large crane, two dip tanks, and a spray nozzle (powered by a 30 kW [40 H.P.] pump). The roots are dipped into the two tanks, sequentially, and then they are rinsed with the nozzle. After the roots are washed, they are loaded onto a walking floor trailer for transport to the dryerlpelletizer.

The washed roots are transported to a first grinding facility, using a Werlor Mill (Werlor Waste Control, Inc., 1420 Ralston Ave., Defiance, Ohio 43512), through which the roots are reduced to a 10 mm grind.

A list of suitable equipment for washing the biomass material is set forth in Table 2.

TABLE 2

| ACTIVITY | EQUIPMENT |
|---|---|
| Washing: The harvested roots are | Large crane with tongs, |
| staged at the washing station. A | two dip tanks (dumpsters), |
| large crane with tongs is used to | 40 HP pump with spray nozzle, |
| dunk the roots in a series of two | Walking Floor Trailer and truck. |
| tanks. The roots are then sprayed | |
| with water from a 40 HP pump for | |
| the final wash step. The washed | |
| roots are staffed and then loaded | |
| on Walking Floor Trailer for | |
| transport to the dryer/pelletizer. | |
| All roots are pre-ground at the | |
| dryer prior to processing. | |

3. Drying the Harvested Material

The washed biomass material is passed through an alfalfa drier to reduce the water content. After the biomass has been dried, it is ground further to a powder.

4. Grinding and Pelletizing

The final grind is wetted with steam or water to obtain a humidity level of about 812%. The grind is pressed into 10 mm diameter pellets, and stored in silos before transportation to a storage warehouse. From there, the material is loaded onto railcars for transport to various taxane extraction facilities.

By pelletizing the biomass, the amount of material that can be loaded onto a railcar or other transportation vehicle approaches the volume and mass limits of the container, thus utilizing the transportation to its maximum benefit. If the biomass were not pelletized, the transportation costs would be substantially higher in some instances.

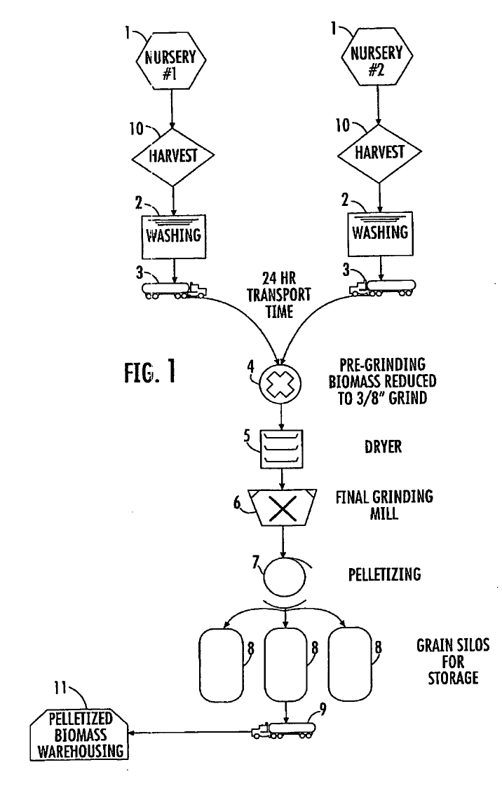

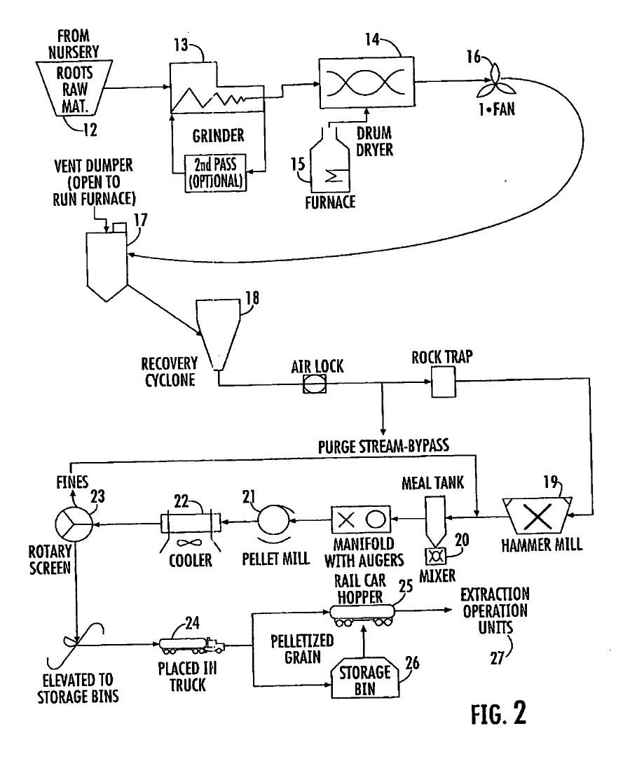

Two schematic, non-limiting diagrams (FIGS. 1 and 2) show alternative embodiments of the present invention. In FIG. 1, Yew trees are harvested from nursery (1) using harvesting equipment (10). The harvested material is then washed using washer (2) and then transported by truck (3) within a 24-hour period. The material is pre-ground to ⅜″ grind (4). The material is dried in dryer (5), and then placed in a final grind mill (6). The ground biomass material is then formed into pellets in pelletizing machine (7). The pellets may be stored in grain silos (8), or transported by truck (9) to an appropriate warehouse (11). In FIG. 2, Yew biomass material (12) is optionally placed in grinder (13). The ground material is then placed in a drum dryer (14). Here, the heat may be supplied by furnace (15) and distributed by fan (16). The material passes through vent dumper (17) and through recovery cyclone (18). The dried material is processed by a Hammer Mill (19) and then mixed using mixer (20). The material is pelletized using pellet mill (21). The pelletized biomass is cooled by cooler (22). The material is sorted by rotary screen (23) and then placed in truck (24) or rail car (25) or stored in storage bin (26). Taxanes are then extracted from the pellets by extraction operation units (27).

As used herein, “taxane or taxane molecule” includes but is not limited to a molecule that contains a basic baccatin III structure with a (2R,3S)—C₆H₅CH(Rx)CH(OH)C(O)— group forming an ester with the hydroxyl group located at the C-13 position of the basic baccatin III structure. The group represented by Rx can be an amino group, a salt of an amino group (e.g., an ammonium salt), an amino group which is protected with an amino protecting group, or a substituent which may be converted into an amino group. Various isomers, homologues, and analogues of the basic baccatin III structure, and of the (2R,3S)—C₆H₅CH(Rx)CH(OH)C(O)group also are included in the definition of a taxane molecule.

Also, a 10-deacetylbaccatin III structure is contemplated within the scope of a taxane molecule. Included within the definition of a taxane or taxane molecule include, but are not limited to, primary taxanes, for example taxol A (paclitaxel), taxol B (cephalomanninc), taxol C, taxol D, taxol E, taxol F, and taxoi G. Further, the definitoin of a taxane or taxane molecule includes docetaxel (TAXOTERE®). (See, e.g., PCT Ser. Appl. No. PCT/US03/105566, which is incorporated herein by reference). As used herein, a “basic baccatin III structure” means a compound having the formula as shown the aforementioned application, where each of R₁, R₂, R₄, R₇, R₁₀ and R₁₃ independently is hydrogen, an alkyl group, an acyl group, an aryl group, an arylalkyl group, a vinyl group, an ether group, an ester group, a glycoside group, an oxo group, or a hydroxyl protecting group. Included within the definition of a basic baccatin III structure is baccatin III, which has the formula as shown in the aforementioned application, and 10-deacetylbaccatin III, which has the formula as shown in the aforementioned application, where Ac is an acetyl group (CH3C(O)—), and Bz is a benzoyl group (PhC(O)— or C₆H₅C(O)—).

In many commercial pelletizing operations, the resulting pellet is a product of a controlled manufacturing process. For example, it is well known that variations in the amount of water or liquid in the raw material will have a marked effect both on the ability to form the material into pellets and on the strength and integrity of the pellets formed. The present invention provides a process that forms a stable Yew pellet for transportation and extraction of taxanes and other natural products contained therein.

The present invention is suitable for pelletizing Yew biomass at different scales, including but not limited to: (i) Laboratory/Pilot Scale Pelletizing Runs; and (ii) IndustrialScale/Large-Scale Pelletizing Runs. A description of each type of run is described below. The Yew pellets produced herein are suitable for extracting taxane molecules from the pellets.

Laboratory/Pilot-Scale Pelletizing Runs

Pelletizing may be carried out on Tcaxiis media x “Densiformis” ground root material. Here, pellets can be made with a California Pellet Mill (“CPM”), Crawfordsville, Ind.. This pelletizing mill comprises dies having ⅛″ and 3/16″ extrusion holes. During the pelletization process, the mill speed may be adjusted to prevent the temperature from exceeding 200° F., preferably less than 180° F., more preferably 140° F. to 160° F. Water can be added to the powdered biomass material before pelletization. Other adjustments to the pellet mill can be made to facilitate the pelletization process, such adjustments are known to those skilled in the art.

Industrial-Scale/Large-Scale Pelletizing Runs

Pellets can be made using a CPM pellet mill. A large CPM pellet mill can pelletize approximately 55 metric tons of Densiformis' roots biomass during a one-day run. The resulting pellets can be ¼ inch in diameter. Preferably the resulting moisture content in the pellets can be approximately 5 to 6 wt % water.

Industrial-Scale/Large-Scale Pelletizing Runs

Pellets can also be made using a Bliss Hammer Mill. The Yew ground material biomass can be dried in a rotating drum and ground further by using this mill. The pelletizing can be done in two Sprout pelletizers with smaller dies. The dies can have ¼″ holes with tapered relief. The throat length for compression can be 1¼″ long. Water can be added to the Yew ground material before pelletization. Each pelletizer mill can process 3,500 lb ground biomass/hr. Each mill may consume 200 A at 460V 3 phase.

Pelletizing Process

The biomass, after reaching the appropriate conditions of size and content, are first harvested. After harvesting, the Yew tree material may be ground by conventional means. Suitable equipment for harvesting the tops of yew plants include those developed by Dr. Peter Felker (Texas A&M University - Kingsville) using a flail harvester (http://www.newuses.orR/EG/EG-19/19harvester.html or http://www.woodycrops.org/mechconf/mclaughl.html).

In one alternative embodiment, the harvesting is directed to the roots of the Taxus plant. After the roots have been removed from the soil, they may be piled on a concrete slab, and washed using a high pressure hose and a shovel or other suitable device to turn the mass over. The roots may be dried through an alfalfa drier and then ground by conventional means. In one embodiment, approximately 5% to 6% w/w water is added to the Yew biomass. The water content should be carefully monitored. Too much water in the pellets could encourage microbial growth, which is undesirable. Also, excess water may adversely affect the taxane extraction process, causing a reduction in the extraction efficiency. In one embodiment, the water content may be controlled by a water or steam bleed steam. This is more desirable.

In another embodiment, the following ranges of water (w/w) may be added to the Yew tree material: from about 0.25% to about 0.5%; about 0.5% to about 1.0%; about 1.0% to 1.5%; 1.5% to about 2.0%; 2.0% to about 3.0%; 3.0% to about 4.0%; 4.0% to about 5.0%; 5.0% to about 6.0%; 6.0% to about 7.0%; about 7.0% to about 8.0%; 8.0% to about 9.0%; about 10.0% to about 11.0%; about 11.0% to about 12.0%; about 12.0% to about 13.0%; about 13.0% to about 14.0%; about 14.0% to about 15.0%; about 15.0% about to 20.0%. Other ranges of water include: about 1% to about 5%; about 5% to about 10%; about 10% to about 15%; about 15% to about 20%.

In one preferred embodiment, the pellets are made using a CPM pellet mill. The speed/capacity of the CPM Pelletizing Mill is affected by the moisture content of the wetted biomass. The normal capacity rating of the CPM laboratory pelletizing mill is about 125 lbs/hr. However, if the moisture content of the wetted biomass is about 30% to about 36% by weight, an additional 30-60 lbs/hr additional capacity may be achieved. If the moisture content of the wetted biomass is too low, it will plug the dies. Conversely, if the materials are too wet, unpelletized material will flush out the die.

The ground solids may be wetted using either water or steam, and then pelletized to a diameter of about 4 to 6 mm ( 3/16 to ¼ inch). This diameter is preferred for further processing the biomass. Smaller diameters may clog the dies, increase the processing temperature, and also increase the wear on the dies. After the pellets are formed, they may be allowed to cool, and then transported. The pellets formed from the process of the present invention show no loss of taxanes over the course of this treatment.

The finished pellets can be stored or used as desired. In the present invention, it is preferred that the pellets be substantially cylindrical or parallelepiped. The maximum cross section of each individual pellet should be about 3/16 to about ¼ inch. While the production of cylindrically shaped pellets is preferred, the invention in its broadest aspects contemplates producing pellets of any suitable configuration. For example, the pellets may be cube-shaped. In one alternative embodiment, the bulk density of pellets produced in the present invention can be at least 30 lbs/ft.³, up to 50 lbs/ft³. In one alternative embodiment, the bulk density of the resulting pellet may include the following ranges: about 30 lbs/ft³ to about 35 lbs/ft³; about 35 lbs/ft³ to about 40 lbs/ft³; about 40 lbs/ft³ to about 45 lbs/ft³; about 45 lbs/ft³ to about 50 lbs/ft.³

Equipment Used for Pelletizing

The following is a list of grinding and pelletizing equipment suitable for pelletizing Yew biomass.
CPM (2975 Arline Circle, Waterloo Iowa)

A large range of suitable industrious equipment is available from this company.
Andritz Sprout-Bauer (Sherman St, Muncy, Pa.)

Hammermills with a range of power (22-255 kW)

Pellet mills with varying power (30-560 kW) and die press area (620-14313 c
Bliss Industries (1415-T W. Summit Ave, Ponca City, Okla.)

Hammermills with a range of power (6-447 kW, 10-600 H. P.)

Pellet mills with varying power (50-300 kW, 80-500 H.P.) and die press area (1500-7800 cm²).

Pellets can be readily made from ground biomass materials with a number of commercially available pelletizing mills. One of the major suppliers of large-scale pelletizing machinery is California Pellet Mill Co, based in Crawfordsville, Ind. A preferred method of pelletizing Yew is described in U.S. Appl. No. 60/437,434, filed Dec. 31, 2002 and PCT Appl. No. , filed Dec. 30, 2003, Both of these applications are incorporated herein by reference in their entirety.

Taxanes Extracted from Yew Pellets

An important component of manufacturing pellitized Yew material is to produce the pellets without reducing the available taxane content inside the pellet, and adversely affecting the taxane extraction process from the pellets. As used herein, “taxane or taxane molecule” include but are not limited to a molecule that contains a basic baccatin III structure with a (2R,3S)—C₆H₅CH(Rx)CH(OH)C(O)— group forming an ester with the hydroxyl group located at the C-13 position of the basic baccatin III structure. The group represented by Rx can be an amino group, a salt of an amino group (e.g., an ammonium salt), an amino group which is protected with an amino protecting group, or a substituent which may be converted into an amino group. Various isomers, homologues, and analogues of the basic baccatin III structure, and of the (2R,3S)—C₆H₅CH(Rx)CH(OH)C(O)— group also are included in the definition of a taxane molecule.

Also, a 10-deacetylbaccatin III structure is contemplated within the scope of a taxane molecule. Included within the definition of a taxane or taxane molecule include, but are not limited to, primary taxanes, for example taxol A (paclitaxel), taxol B (cephalomannine), taxol C, taxol D, taxol E, taxol F, and taxol G. Further, the definition of a taxane or taxane molecule includes docetaxel (TAXOTERE®). (See, e.g., PCT Ser. Appl. No. PCT/US03/105566, which is incorporated herein by reference). As used herein, a “basic baccatin III structure” means a compound having the formula as shown in the aforementioned application, where each of R1, R2, R4, R7, R10 and R13 independently is hydrogen, an alkyl group, an acyl group, an aryl group, an arylalkyl group, a vinyl group, an ether group, an ester group, a glycoside group, an oxo group, or a hydroxyl protecting group. Included within the definition of a basic baccatin III structure is baccatin III, which has the formula as shown in the aforementioned application, and 10-deacetylbaccatin III, which has the formula as shown in the aforementioned application, where Ac is an acetyl group (CH₃C(O)—), and Bz is a benzoyl group (PhC(O)— or C₆H₅C(O)—).

In one alternative embodiment, the present invention is directed to a process for making Yew pellets comprising the following steps: (a) adding from about 1% to about 20% w/w water to ground Yew tree material; and (b) compressing the wetted Yew tree material in a die to form a pellet whereby by the resulting pellet has a final moisture content less than 10% by weight. In one alternative embodiment, the Yew tree material is ground into a powder. In another embodiment, the Yew material is derived from roots.

In another alternative embodiment, the process of the present invention provides a resulting pellet having a bulk density from about 30 lbs/ft³ to about 50 lbs/ft³. In another embodiment, the present process provides a resulting pellet having a final diameter from about 1/16 inch to about 1 inch, or from about 3/16 inch to about ¼ inch or the final diameters ¼ inch.

In another embodiment, in carrying out the present process the diameter of the extrusion holes of the die is from about 1/16 inch to about 1 inch, or from about 3/16 inch to about ¼ inch. Further, in another embodiment, the temperature of the resulting pellet as it emerges from the die is from about 100° F. to about 212° F., or from about less than 180° F. or from about 130° F. to about 160° F.

In another alternative embodiment, the present invention is directed to a Yew pellet having a final moisture content less than 10% by weight. In another embodiment, the pellet has a final moisture content from about 1% to about 6% by weight, or from about 4% to about 6% by weight. In another embodiment, the pellet of the present invention has a final bulk density from about 30 lbs/ft³ to about 50 lbs/ ft3, In another embodiment, the diameter of the pellet is from about 1/16 inch to about 1 inch or 3/16 inch to about ¼ inch or ¼ inch.

Turning the pellet mill to higher throughput can reduce the quality of the pellets. Also, as the thickness of the die is increased, the resulting pellets become harder and the capacity and ability of the mill to run are adversely affected. In one alternative embodiment, the die is made of high carbon steel. Carbon steel is more preferable than chrome or stainless steel dies.

The use of additives can be important with powered biomass materials. In the case of ground root biomass, the use of additives is less important, and in some instance is not required. With Yew tops, for example, it is advantageous to use binder additives to facilitate production of pellets suitable for extraction. Such binders may include, but are not limited to, guar gum, starches, vegetable oils, alginates, xanthan gums, locust bean gum and other suitable binders.

Throughout the description herein, where the present invention is described as having, including, or comprising specific components, or where processes are described as having, including, or comprising specific process steps, it is contemplated that the present invention also consists essentially of, or consists of, the recited components or processing steps. Further, it should be understood that the steps or order of performing certain actions or steps are immaterial so long as the invention remains operable. Moreover, two or more steps or actions may be conducted simultaneously so long as the invention remains operable. Also, one or more steps or components may be omitted from the claimed invention, so long as the performance of the invention does not depart from the spirit or essential characteristics thereof.

The invention is further demonstrated by the following examples. The examples are for purposes of illustration and is not intended to limit the scope of the present invention.

### EXAMPLES

### Example 1

### Pelletizing Process

1. 2000 grams of dry ground Yew tree root biomass (powder).
2. 200 g water added to the Yew biomass.
3. The biomass and water were mixed in a HOBART blender with eccentric drive/paddle.
4. The die is first purged with oats, forming pelletized oats.
5. The wetted biomass was run through a CPM ‘CL3’ Laboratory pellet mill. This m had a 3 HP motor (220 Volts). The temperature of the resulting pellets wereapproximately 160° F. The pellets were dark in color, ranging in length of about ½ inch to 1 inch.
6. After 20 minutes, the moisture content of the pellets was approximately 10.3%. After 30 minutes, it was approximately 10.4%. The bulk density of the pellets was 39 lb./ft³* The bulk density of the biomass powder was approximately 18 Ibs/ ft3, Capacity tests were run on the pellet mill to determine the mill's capacity for the biomass material. For example, to produce ⅛ inch pellets, the capacity for the mill was 117 lbs/hr.

### Example 2

### Additives

In some instances, additives can be used to facilitate production of pellets suitable for taxane extraction. The following examples describe an additive (Guar Gum) that can be added to the wetted biomass. Each pelletizing run followed the general protocol shown in Example 1, To evaluate the amount of fines, the die was heated beforehand.

b 1% Guar Gum

Guar gum run: 3/16″ pelletizing; 2000 g of ground Yew root biomass; 200 g H₂O; 20 g guar gum;

Guar Gum Source—*non-food grade Rawtec UFF-MV (5/21/01 RX 1036)

Temperature higher: more fines in 155° F.; 128 lb/lir

Bulk density: 40 lb/ft³; and

Wgt. fines 208 g; wgt pellets 1822 g; 10.2% fines; 90% conversion.

1/2% Guar Gum

Guar gum run: ⅛″ pelletizing;

2000 g of ground Yew root biomass; 200 g H₂O;

10 g guar gum (=½%) (added as powder);

Temp 150° F. to 163° F.;

Rate: 136 lb/hr.;

Wgt. pellets: 1894 g; wgt fines. 139 g; or 7% fines; and

Bulk density: 35 lb/ft³.

2% Guar Gum

Guar gum run: ⅛″ pelletizing;

2000 g ground Yew root biomass; 200 g H₂O; 40 g guar gum (=2%) (added as powder); Temp 162° F. to 174° F.;

Rate Capacity: 145 lb/hr.

Fines 140 g; pellets 193° g; 7% fines; Bulk density: 37 lb/ft³.

Control Run (No Guar Gum Added)

2000 g ground Yew root biomass; 200 g H₂O; ⅛″ pelletizing;

Temp: 148° F. to 170° F.;

Rate: capacity 95 lb/hr;

Wgt fines: 95 g; wgt of pellets 2000 g; 4.5% fines; and

Bulk density: 34 lb/ft3,

### Example 3

### Extraction of Taxanes From Pellets

A series of experiments were run to confirm that paclitaxel and other taxanes survive the pelletizing process of the present invention. Specifically, Yew pellets were extracted using hot methanol in a Soxhlet apparatus: ⅛″ pellets (no additives), 3/1″ pellets (no additives), and non-pelletized biomass powder (from which the pellets were made). Table 3 shows the recovery of taxanes.

TABLE 3
Soxhlet Thimble Wgt. Grams

| Exp. Run No. | Tare | Gross | Mass of Biomass |
|---|---|---|---|
| 1 | 12.5 | 47.8 | 35.3 g starting |
| | | | material |
| 2 | 13.2 | 50.7 | 37.5 g dry ground |
| | | | biomass |
| 3 | 14.3 | 73.8 | 59.5 g ⅛″ pellets, |
| | | | no additives |
| 4 | 13.0 | 68.2 | 55.2 g ⅛″ pellets, |
| | | | no additives |
| 5 | 12.8 | 74.7 | 61.9 g 3/16″ |
| | | | pellets, no additives |
| 6 | 11.1 | 70.2 | 59.1 g 3/16″ |
| | | | pellets, no additives |

HPLC analysis showed good recovery of total taxanes approximately (approximately 0.02% w/w) for both pellets and non-pellets. HPLC analysis also showed good recovery of paclitaxel (approximately 0.01% w/w) for both pellets and non-pellets. These tests showed no significant loss/degradation of extractable paclitaxel as a result of the pelletizationprocess.

The invention, as described above, may be varied in certain aspects. For example, pellet production may be accomplished continuously or in separate batches. In addition, operating parameters may be varied, depending on many factors. These factors include available materials, economic considerations and energy needs of specific consumers. However, it is to be understood that these and other factors can be made by those skilled in the art without distracting from the sprit and scope of the invention.

Throughout the description, where the present invention is described as having, including, or comprising specific components, or where processes are described as having, including, or comprising specific process steps, it is contemplated that the present invention also consists essentially of, or consists of, the recited components or processing steps. Further, it should be understood that the order of steps or order for performing certain actions are immaterial so long as the invention remains operable. Moreover, two or more steps or actions may be conducted simultaneously so long as the invention remains operable. Also, one or more steps or elements may be omitted from the claimed invention, or the invention described herein suitably may be practiced in the absence of any component or step which is not specifically disclosed herein, so long as the invention remains operable.

Further, the present invention may be embodied in other specific forms without departing from the spirit or essential characteristics thereof. The foregoing embodiments are therefore to be considered illustrative rather than limiting the invention described herein.

The content of each patent and non-patent document referred to herein is expressly incorporated herein by reference in its entirety.

We claim:

1. A process of making Yew pellets from grounded Yew tree material comprising the following steps:
a. adding from about 1% to about 20% w/w water to the ground Yew tree material;
b. compressing the Yew tree material in a die to form a pellet whereby by the resulting pellet has a final moisture content less than 10% by weight.

2. The process according to claim 1, wherein the resulting pellet has a final moisture content less than 8% by weight.

3. The process according to claim 1, wherein the resulting pellet has a final moisture content from about 1% to about 6% by weight.

4. The process according to claim 1, wherein the resulting pellet has a final moisture content from about 4% to about 6% by weight.

5. The process according to claim 1, wherein the final bulk density of the resulting pellet is from about 30 lbs/ft³ to about 50 lbs/ft³

6. The process according to claim 1, wherein the final diameter of the resulting pellet is from about 1/16 inch to about 1 inch.

7. The process according to claim 1, wherein the final diameter of the resulting pellet is from about 3/16 inch to about ¼ inch.

8. The process according to claim 1, wherein the final diameter of the resulting pellet is ¼ inch.

9. The process of claim 1, wherein the diameter of the extrusion holes of the die is from about 1/16 inch to about 1 inch.

10. The process of claim 1, wherein the diameter of the extrusion holes of the die is from about 3/16 inch to about 11/4 inch.

11. The process of claim 1, wherein the temperature of the resulting pellet as it emerges from the die is from about 100° F. to about 212° F.

12. The process of claim 1, wherein the temperature of the resulting pellet as it emerges from the die is from about less than 180° F.

13. The process of claim 1, wherein the temperature of the resulting pellet as it emerges from the die is form about 130° F. to about 160° F.

14. A Yew biomass pellet produced by a process comprising the following steps:
a. adding from about 1% to about 20% w/w water to ground Yew tree material; and
compressing the wetted Yew tree material in a die to form a pellet whereby by the resulting pellet has a final moisture content of less than 10% by weight.

15. A Yew biomass pellet having a final moisture content of less than 8% by weight.

16. The Yew biomass pellet of claim 15, wherein the pellet has a final moisture content from about 1% to about 6% by weight.

17. The Yew biomass pellet of claim 15, wherein the pellet has a final moisture content from about 4% to about 6% by weight.

18. The Yew biomass of claim 15, wherein the bulk density of the pellet is from about 30 lbs/ft³ to about 50 lbs/ft³

19. The Yew biomass of claim 15, wherein the diameter of the pellet is from about 1/16 inch to about 1 inch.

20. The Yew biomass pellet of claim 15, wherein the diameter of the pellet is from about 3/16 inch to about ¼ inch.

21. The process according to claim 1, wherein the final diameter of the resulting pellet is ¼ inch.

22. A method of harvesting Yew biomass material, comprising the steps of:
i) harvesting the tops or roots of the Yew tree;
ii) washing the harvested tops or roots;
iii) drying the harvested tops or roots;
iv) grinding the harvested tops or roots into a fine powder.
